(19) United States
(12) Patent Application Publication
(10) Pub. No.: US 2011/0077645 A1
(43) Pub. Date: Mar. 31, 2011

(54) Electrosurgical Pencil with Synchronous Evacuation Function

(76) Inventor: San-Chih Yang Lin, Keelung City (TW)

(21) Appl. No.: 12/568,727

(22) Filed: Sep. 29, 2009

(51) Int. Cl. A61B 18/18

(52) U.S. Cl. 606/45

(57) ABSTRACT

An electrosurgical pencil with synchronous evacuation function includes a pencil body defined with a first containing space and a second containing space therethrough. The first containing space is adapted to contain an electropencil. The second containing space is adapted to be an evacuation passage. A first front end of the electropencil is mounted with a replaceable electrode. A first rear of the electropencil is adapted to connect to an electric wire connected to a current generator. A second front end of the second containing space is integrally formed a tubular body having an opening. A second rear end of the second containing space is mounted with a connector. The connector is connected to a suction instrument with a negative pressure via a soft tube. When the end portion of the electrode operates, the opening of the evacuating passage draws smoke and blood at the same time.

### BACKGROUND OF THE INVENTION

1. Field of the Invention

The present invention relates to an electrosurgical pencil, and more particularly to an electrosurgical pencil with synchronous evacuation function.

2. Description of Related Art

During surgery, surgeons may use an electrotome to cut the tissue, including muscle tissue, lymphatic tissue, and blood vessels, etc. Sometimes massive bleeding could happen during surgery and it might result in difficulty in performing the operation because the blood may make the operation field obscure. The electrosurgical pencil can be adapted to coagulate bleeding according to the principles of high frequency current. Therefore, electrosurgical pencil is provided in the surgery for cutting tissue and stopping bleeding (coagulation).

Surgical smoke and aerosol are typically produced during procedures utilizing electrosurgical pencil. The smoke and aerosol can occlude the vision of the surgeon and the rest of team. The electrosurgical smoke is potentially hazardous if inhaled by the operation personnel. If the smoke is not evacuated in time, it may be inhaled. Thus, when the surgeon utilizes the electrosurgical pencil to cut tissue or stop bleeding during surgery, the surgeon needs another assistant or nurse to hold a suction device, including a metal tip and a tube, to evacuate the smoke, remove the blood clots, and stop bleeding. Furthermore, the surgeon and his assistant (or scrubing nurse) have to cooperate for a long period of time to become familiar before they can cooperate and perform well. Therefore, the conventional electrosurgical pencil results in a difficult operation and surgical labor waste.

Moreover, the smoke may occlude the vision, lengthen the procedure, and hamper the performance of the electrosurgical pencil. The smoke could be harmful to the whole team of operation, including surgeon and the rest of team, if inhaled. Conventional metallic suction tip is connected to a soft tube that is connected to a suction instrument equipped with a motor. The suction instrument typically is mounted in the operating room for evacuating smoke and blood. Thus, the need for improvement still exists.

### SUMMARY OF THE INVENTION

The main objective of the present invention is to provide an improved electrosurgical pencil with synchronous evacuation function includes a pencil body defined with a first containing space and a second containing space therethrough. The first containing space is adapted to contain an electropencil. The second containing space is adapted to be an evacuation passage. A first front end of the electropencil is mounted with a replaceable electrode include blade, extended blade, needle, extended needle, and ball configurations. A first rear end of the electropencil is adapted to connect to an electric wire connected to a current generator. A second front end of the second containing space is integrally formed a tubular body having an opening. The opening closes to an end portion of the electrode. A second rear end of the second containing space is mounted with a connector. The connector is connected to a suction instrument with a negative pressure via a soft tube; wherein when the electrode is used to cut tissue, the opening of the evacuating passage draws smoke and blood at the same time.

By utilizing the invention, the following advantages are obtained. First, the present invention provides an electrosurgical pencil with synchronous evacuation function, the surgeon doesn't need another assistant or nurse to hold a suction tube to evacuate the smoke. Thus, the present invention can effectively prevent surgical labor from wasting. Second, the opening of suction tubular body is mounted close to the electrode of electrosurgical pencil. Therefore, surgical smoke can be evacuated quickly to keeping the surgical team healthy because the surgical smoke may contain toxic components.

Third, the surgical smoke that is created during surgical may occlude the vision to lengthen the procedure and hamper the performance of the electrosurgical pencil. Therefore, combined suction tube and electrosurgical pencil apparatus proposed in the present invention can effectively solve the problem of being lack of visibility and to improve the efficiency of the surgery.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of an electrosurgical pencil with synchronous evacuation function of a first embodiment in accordance with the present invention;

FIG. 2 is a sectional view of the electrosurgical pencil with synchronous evacuation function of the first embodiment in accordance with the present invention;

FIG. 3a is a partial perspective view of an electrosurgical pencil with synchronous evacuation function of the first embodiment in accordance with the present invention;

FIG. 3b is a partial perspective view of an electrosurgical pencil with synchronous evacuation function of a second embodiment in accordance with the present invention;

FIG. 4a is a partial perspective view of an electrosurgical pencil with synchronous evacuation function of a third embodiment in accordance with the present invention;

FIG. 4b is a partial perspective view of an electrosurgical pencil with synchronous evacuation function of a fourth embodiment in accordance with the present invention;

FIG. 5a is a partial perspective view of an electrosurgical pencil with synchronous evacuation function of a fifth embodiment in accordance with the present invention;

FIG. 5b is a partial perspective view of an electrosurgical pencil with synchronous evacuation function of a sixth embodiment in accordance with the present invention; and

FIG. 6 is a perspective view to show various kinds of hard connectors in accordance with the present invention.

### DETAILED DESCRIPTION OF THE INVENTION

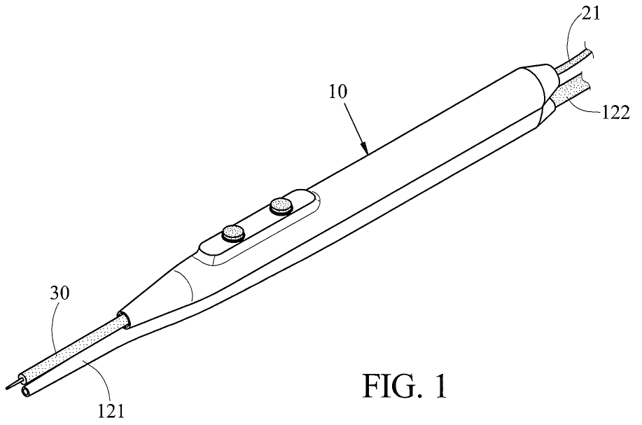

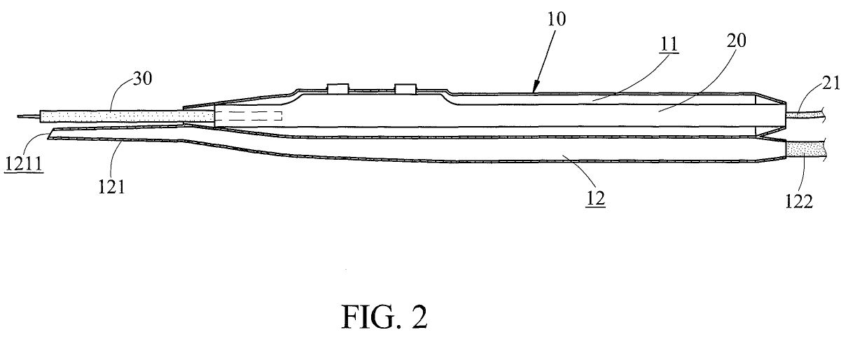

Referring to FIGS. 1-5b, an electrosurgical pencil with synchronous evacuation function includes a pencil body 10 defined with a first containing space 11 and a second containing space 12 therethrough. The first containing space 11 is adapted to contain an electropencil 20. The second containing space 12 is adapted to be an evacuation passage. A first front end of the electropencil 20 is mounted with a replaceable electrode 30. A first rear end of the electropencil 20 is adapted to connect to an electric wire 21 connected to a current generator. A second front end of the second containing space 12 is integrally formed a tubular body 121 having an opening 1211. The opening 1211 is close to an end portion of the electrode 30. A second rear end of the second containing space 12 is mounted with a connector 122. The connector 122 is connected to a suction instrument with a negative pressure via a soft tube 40. When the end portion of the electrode 30 is operated, the opening 1211 of the evacuating passage draws smoke and blood at the same time. Thus, the surgeon does not need another assistant or nurse to hold a suction tube to evacuate the smoke and stop bleeding (coagulation). Moreover, the present invention can effectively prevent surgical labor from wasting and surgical smoke can be evacuated quickly for keeping the surgical team healthy.

The first containing space 11 and the second containing space 12 do not communicate with each other.

During surgery, a patient has to be stuck with an electrode sticker on his foot. The electrode sticker is electrically connected to an instrument for producing current. Thus, combined electropencil and electrode sticker apparatus can be adapted to cut and stop bleeding.

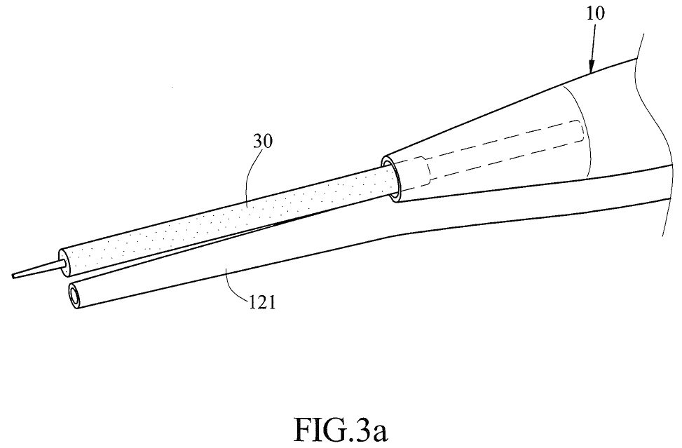

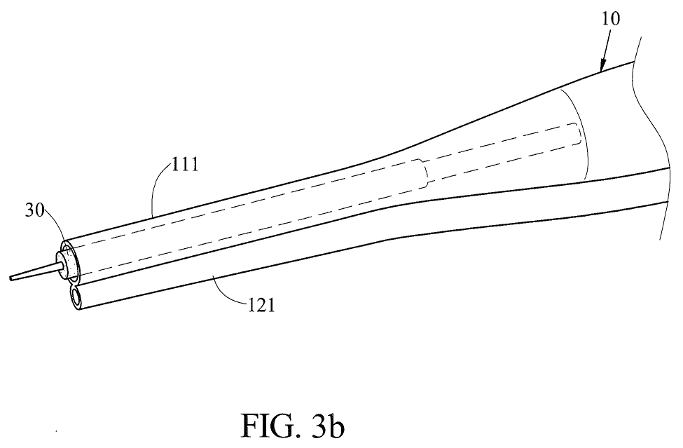

Preferably, the electrode 30 is directly connected to the electropencil 20 in the first containing space 11 (as shown in FIG. 3a).

Preferably, a third front end of the first containing space 11 is integrally formed with a connecting tube 111; the electrode 30 is connected to the electropencil 20; and the electrode 30 is covered and mounted in the connecting tube 111 (as shown in FIG. 3b).

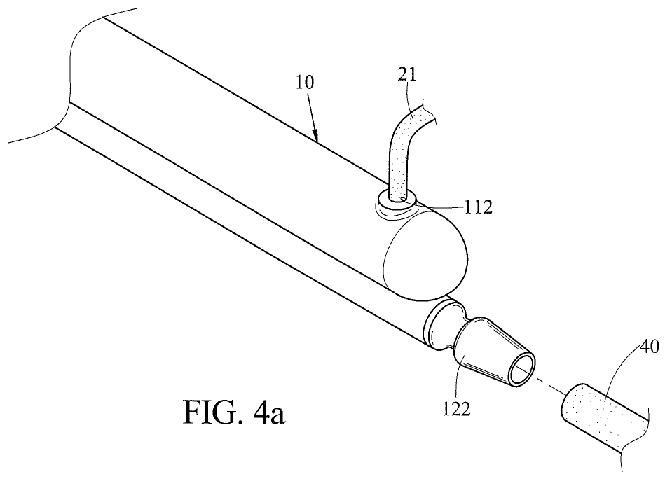

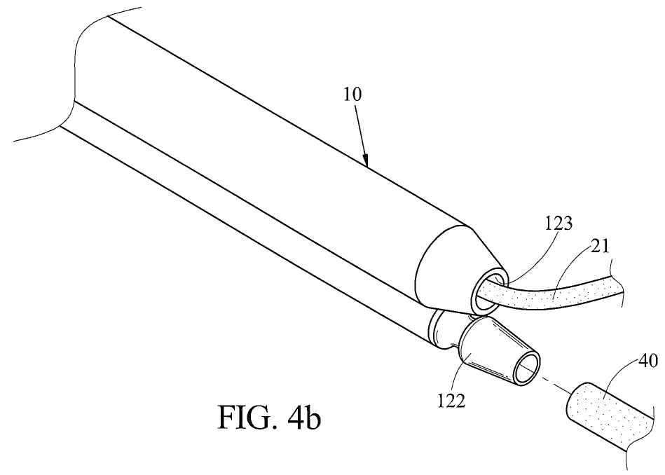

Preferably, a third rear end of the first containing space 11 is provided with a hole 112 formed at a side of the pencil body 10; the hole 112 is provided for the electric wire 21 extending from; and the connector 122 is connected to the soft tube 40 (as shown in FIG. 4a).

Preferably, a third rear end of the first containing space 11 is provided with a hole 123 for the electric wire 21 extending from and the connector 122 is connected to the soft tube 40 (as shown in FIG. 4b).

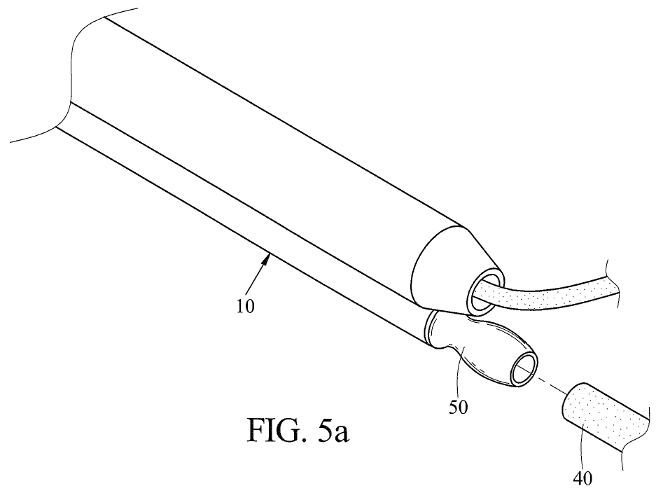

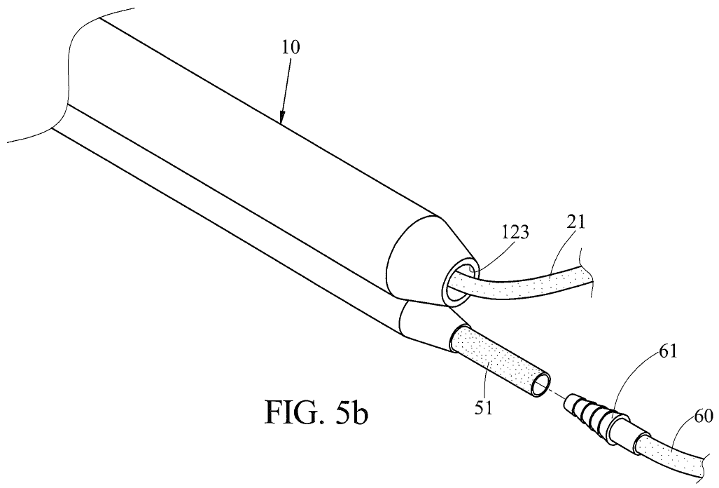

Preferably, the second rear end of the second containing space 12 is mounted with a hard connector 50 connected to a soft tube 40 and the harder connector 50 is directly connected to the soft tube 40 (as shown in FIG. 5a).

Preferably, the second rear end of the second containing space 12 is mounted with a soft connector 51 connected to a soft tube 60 and the soft tube 60 has a taper and protruding portion 61 for preventing the soft tube 60 from coming off (as shown in FIG. 5b).

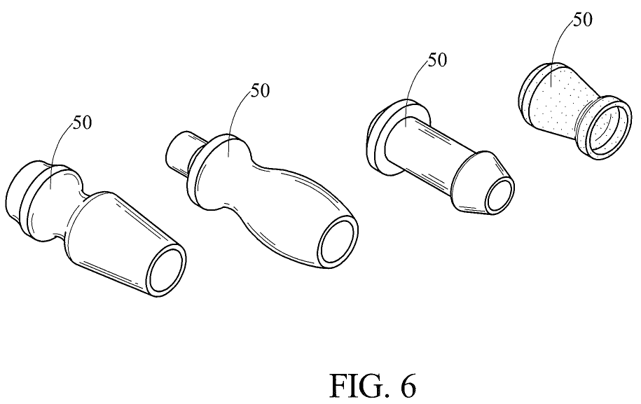

Preferably, the opening 1211 of the tubular body 121 includes an inclined plane or a plane. Various kinds of hard connectors 50 can be used in accordance with the present invention (as shown in FIG. 6).

By utilizing the invention, the following advantages are obtained. First, the present invention provides an electrosurgical pencil with synchronous evacuation function and the surgeon does not need another assistant or nurse to hold a suction tube to evacuate the smoke. Thus, the present invention can effectively prevent surgical labor from wasting. Second, the opening of suction tubular body is mounted close to the electrode. Therefore, surgical smoke can be evacuated quickly for keeping the surgical team healthy because the surgical smoke may contain harmful toxic components. Third, smoke generated during surgical may block vision to adversely increase procedure time and decrease the performance of the electrosurgical pencil. Therefore, the combined suction tube and electrosurgical pencil apparatus proposed in the present invention can effectively solve the problem of poor visibility and increase the efficiency of the surgery.

Although the invention has been explained in relation to its preferred embodiment, it is to be understood that many other possible modifications and variations can be made without departing from the spirit and scope of the invention as hereinafter claimed.

1. An electrosurgical pencil with synchronous evacuation function, comprising:
a pencil body defined with a first containing space and a second containing space therethrough, the first containing space being adapted to contain an electropencil and the second containing space being adapted to be an evacuation passage wherein a first front end of the electropencil is mounted with a replaceable electrode, a first rear end of the electropencil is adapted to connect to an electric wire connected to a current generator, a second front end of the second containing space is integrally formed with a tubular body having an opening, the opening being proximate an end portion of the electrode, and a second rear end of the second containing space is mounted with a connector, the connector being connected to a suction instrument with a negative pressure via a soft tube; and wherein when the end portion of the electrode is operated, the opening of the evacuating passage draws smoke and blood at the same time.

2. The electrosurgical pencil of claim 1, wherein the electrode is connected to the electropencil in the first containing space.

3. The electrosurgical pencil of claim 1, wherein a third front end of the first containing space is integrally formed with a connecting tube; the electrode is connected to the electropencil; and the electrode is covered and mounted in the connecting tube.

4. The electrosurgical pencil of claim 1, wherein a third rear end of the first containing space is provided with a hole formed at one side of the pencil body, the hole being provided for the electric wire extending therefrom; and the connector is connected to the soft tube.

5. The electrosurgical pencil of claim 1, wherein a third rear end of the first containing space is provided with a hole for the electric wire extending therefrom and the connector is connected to the soft tube.

6. The electrosurgical pencil of claim 1, wherein the second rear end of the second containing space is mounted with a hard connector connected to a soft tube.

7. The electrosurgical pencil of claim 1, wherein the second rear end of the second containing space is mounted with a soft connector connected to a soft tube, the soft tube having a taper and protruding portion for preventing the soft tube from coming off.

8. The electrosurgical pencil of claim 1, wherein the opening of the tubular body includes an inclined plane.

9. The electrosurgical pencil of claim 1, wherein the opening of the tubular body includes a plane.
